(19) United States
(12) Patent Application Publication
(10) Pub. No.: US 2026/0196988 A1
(43) Pub. Date: Jul. 9, 2026

(54) METHOD FOR GENERATING ELECTROMAGNETIC FIELDS AND A DEVICE HAVING SUCH A DEVICE

(71) Applicant: Petra FALKE, Donaueschingen (DE)

(72) Inventors: Petra FALKE, Donaueschingen (DE); Jörg HOFMANN, Huefingen (DE)

(21) Appl. No.: 19/130,710

(22) PCT Filed: Nov. 16, 2023
(86) PCT No.: PCT/EP2023/082124

§ 371 Date: Jun. 11, 2025

(30) Foreign Application Priority Data
Nov. 18, 2022 (DE) 10 2022 130 619.4

(51) Int. Cl. H03K 3/017; A61N 2/02; H01F 7/20

(52) U.S. Cl. CPC H03K 3/017; A61N 2/02; H01F 7/20

(57) ABSTRACT

The present invention relates to the generation of electromagnetic fields by an applicator (5) with low-frequency current pulses (10) by a generator (3) which generates the low-frequency current pulses (10) for generating the electromagnetic fields, wherein a plurality of current pulses (10) follow a signal curve, wherein the respective current pulse (10) comprises a first signal component in the form of a rectangular pulse, wherein a current pulse pause (16) is provided between two current pulses (10), and wherein the amplitude of the current pulses (10) is modulated with a modulation frequency in a sinusoidal signal curve. Furthermore, the present invention relates to a device for carrying out the method.

The present invention relates to a method for generating electromagnetic fields by means of low-frequency current pulses, in particular for treating regions of the body, with the features of claim 1, and to an device with the device for generating the low-frequency current pulse with the features of claim 17.

Methods for generating electromagnetic fields are known from the prior art in various configurations and are widely used to apply electromagnetic fields to regions of the body of living beings. The electromagnetic fields generated are used in the state of the art to influence biological processes in the body regions of living beings. An example of such an device is known from publication EP 152 963A1 .

Furthermore, EP 0 594 655 B1 discloses an device with a generator producing a low-frequency and pulsed electric current and a transmitting coil connected to it, whereby the electromagnetic fields produced by the coil are used to apply a current to a body region to be treated. The device taught there is designed to transport ions and protons in particular by specifically influencing the concentration of ions in any part of the body in humans and animals.

The devices known from the state of the art have proven themselves in the past, but there is a desire to further develop this technology in order to improve the mode of action.

This is where the present invention comes in.

Based on this state of the art, the present invention sets itself the task of proposing an expediently improved method for generating electromagnetic fields by means of low-frequency current pulses, in particular for treating body regions of living beings, which expediently eliminates disadvantages from the state of the art.

This task is solved by a method comprising the features of claim 1 and a device for carrying out the method comprising the features of claim 17.

Further advantageous embodiments of the present invention are given in the dependent claims.

The method according to the invention for generating electromagnetic fields with the features of patent claim 1 by means of an applicator with a transmitting coil energized with low-frequency current pulses, in particular for treating body regions of living beings, in particular humans or animals, is characterized in that the current pulses have a signal curve and the respective current pulse comprises a first signal component whose amplitude is formed in the form of a rectangular pulse. A current pulse pause is also provided between two current pulses. According to the invention, the amplitude of the current pulses is modulated with a, preferably single, modulation frequency in a sinusoidal signal curve.

The first signal component within the meaning of the present invention preferably has an approximately constant amplitude over the course of the respective current pulse.

The present invention is based on the idea of generating pulsating electromagnetic fields which become more intense and then weaker over time. The sinusoidal modulated signal curve contributes to the fact that the amplitude of the current pulses rises and falls gently over time along a complete period, whereby a stimulation is achieved in the vegetative system and the ion transport in the applied body region is accomplished in a particularly effective manner. It has also been shown that the sinusoidal amplitude modulation of the current pulses improves the penetration depth of the electromagnetic fields into the body region.

The pulsating electromagnetic fields are used for the treatment, prevention and/or aftercare of sleep disorders, exhaustion, stress, burn-out, pain, degenerative diseases, inflammation, bone fractures, improvement of wound healing, bone fracture healing, wound healing, circulatory disorders, metabolic disorders, performance enhancement and improvement of regeneration in sports, especially competitive sports. The corresponding body region is exposed to the pulsating electromagnetic fields. For example, the above-mentioned applicator can be positioned on the body in such a way that the pulsating electromagnetic fields can affect the area of the body to be treated.

According to a preferred further development of the present invention, the signal curve comprises at least one current pulse sequence which describes at least one complete sinusoidal period. In particular, it has proven to be advantageous if a current pulse sequence describes several periods, although it should be noted here that the number of periods does not necessarily have to be an integer. Accordingly, a current pulse sequence can last for 3.5 periods, for example. It should also be noted that it can also be advantageous if a current pulse sequence comprises a half sinusoidal period.

Furthermore, according to a further development of the present invention, the respective current pulse has a duration of between 0.1 ms and 10 s. In particular, it is preferred if the respective current pulse has a duration of approx. 0.5 ms to approx. 2 ms, with the current pulse having a duration of approx. 2 ms being even more preferred.

The duration of the corresponding current pulse determines in particular a high rate of change of the current in the signal curve. As a result, the induced voltage pulses cannot have a significant phase shift compared to the current pulses. This results in a continuous induction due to the changing electromagnetic field in the body region.

A further embodiment of the present invention provides that the complete sinusoidal period comprises at least four, more preferably at least eight, and still more preferably at least twelve current pulses, wherein preferably the current pulse pauses between the individual current pulses can be of equal length. It should be noted at this point that the number of current pulses per complete sinusoidal period can be increased as required. The number of current pulses depends, among other things, on the modulation frequency, which determines the duration of a current pulse sequence.

A further development of the present invention provides that the signal curve comprises at least three current pulse sequences with approximately the same amplitude. In particular, a current pulse sequence comprising a complete period can also be formed by a half period with a current pulse sequence pause of half a period.

Furthermore, it has proven to be advantageous if the current pulse pause between two current pulses has a duration of between 0.1 ms and 10 s. In particular, it has proven to be advantageous if the current pulse pause is less than approx. 5 ms. In particular, it has proven to be advantageous if the current pulse pause is less than approx. 5 ms and it is even more preferred if the current pulse pause is approx. 0.25 ms.

In addition, it can be advantageous if the current pulses are generated with a carrier frequency. The carrier frequency is preferably between 100 Hz and 100 KHz.

An additional further development of the present invention provides that the duration of current pulses in conjunction with current pulse pauses forms a frequency which is tuned to a mechanical resonance in organs, tissues, cell assemblies or molecules. The mechanical resonance frequency is preferably between 3 Hz and 3 KHz.

For example, 200 Hz resonates with arterioles and causes them to vibrate slightly. This reduces the coefficient of friction of the blood suspension on the endothelium, which can lead to a higher flow velocity, resulting in improved thermoregulation and increased erythrocyte transport to the capillary system.

The respective current pulse can be formed from a superposition of the first signal component already described and a second signal component, whereby the second signal component is formed from a rising and/or falling current. The first signal component and the second signal component are preferably synchronized and are superimposed. Furthermore, the second signal component can advantageously be formed from an increasing and/or decreasing current, which can correspond to the form of a linear, exponential function and/or a Fibonacci number sequence.

At this point, it should be noted that the second signal component can also be described as staircase-shaped. The individual steps can, for example, be due to the carrier frequency and can also correspond to a series of rectangular pulses with increasing and/or decreasing amplitude.

For example, the second signal component can be either increasing or decreasing over the duration of the corresponding current pulse. For example, if the current pulse has a duration of 2 ms, it is conceivable that the amplitude in the second signal component increases for 0.9 ms and then decreases for the remaining 1.1 ms. The ratio between the increasing and decreasing portion can preferably be between 10:1 and 1:10.

Due to a high rate of change of the current in the second signal component with the increasing and/or decreasing function, the induced voltage pulses in particular do not exhibit any significant phase shift compared to the current pulses, resulting in a continuous induction due to the changing electromagnetic field in the body region.

Furthermore, it is preferred if, according to a further development, a current pulse sequence pause is provided between two current pulse sequences at regular or irregular intervals, and that the current pulse sequence pause has a duration that is preferably longer than 0.1 ms and preferably shorter than 10 s. During the current pulse sequence pause, the organism is given the opportunity to allow the biochemical-physical processes stimulated by the pulses to take effect.

According to a preferred further development of the present invention, all current pulses over at least one current pulse sequence have an amplitude which is selected in such a way that the current pulses do not change polarity in the signal curve. In other words, the amplitude of a current pulse sequence can be A(t)≥0 or alternatively A(t)≤0.

In particular, it has proven to be advantageous if all current pulses have an amplitude of A>0 or A<0 over at least one current pulse sequence. Accordingly, the organism is continuously energized during a current pulse sequence, whereby the charged particles are continuously pushed in one direction.

A preferred further development of the present invention provides that the modulation frequency is between 0.5 Hz and 120 Hz. A particularly preferred further development of the present invention provides that the generator can alternate between at least two modulation frequencies, in particular preferably the generator can generate the modulation frequencies approx. 6 Hz, approx. 10 Hz and approx. 16 Hz. These modulation frequencies are adapted to the vegetative system of a human brain, whereby the modulation frequency of approx. 6 Hz stimulates the vegetative system towards the resting state of the human and approx. 16 Hz stimulates the vegetative system towards the physically active human. The third modulation frequency mentioned above of approx. 10 Hz should be emphasized here, whereby this modulation frequency corresponds to a stimulation of the vegetative system to a state of relaxation. Part of the brain of all living beings, in particular the autonomic nervous system, resonates with the modulation frequency and can therefore be stimulated particularly well with the modulation frequency. In particular, it can be advantageous if the modulation frequency-preferably the only one-or at least one of the at least two interchangeable modulation frequencies described above is approximately 2.2 Hz, 7.83 Hz and/or 14.2 Hz. The frequencies mentioned can be harmonic or resonance frequencies of the vegetative system. It should be noted that “approximately” in this context means a tolerance of approx. ±2Hz. Furthermore, the tolerance of the modulation frequency is preferably ±10%.

A further development of the present invention provides that the respective current pulse has a carrier frequency between 100 Hz and 100 KHz or between 50 MHz and 250 MHz. In particular, it is preferable if the carrier frequency is 150 MHz, whereby, together with the sinusoidal amplitude modulation of the current pulses, a focused effective field and a deep penetration depth can be achieved with a high degree of energy transmission. In particular, the combination avoids an undesirable skin effect, in which high-frequency electromagnetic fields only penetrate the surface of the body regions and generate unwanted eddy currents, which can lead to a heat or pain stimulus, for example.

A further development of the present invention also provides that at least one control parameter can be provided which can influence the amplitude of the current pulses, the modulation frequency, the modulation amplitude, the pause duration, a duration of the current pulse sequence pause and/or, a duration of the current pulse sequence or number of periods per current pulse sequence. Typically, such a control parameter may include, for example, biofeedback, a blood pressure monitor, temperature detection, pulse detection or the like, whereby the device can provide a signal curve of the current pulses adapted to the body. In the simplest case, the control parameter input can be formed by an HMI (human-machine interface), such as one or more control elements. However, the control parameter input can also include an interface that is set up to communicate with a measuring tool. Such a measuring tool can be a conventional measuring device, a smart device and/or a “wearable” such as a smartwatch. Such devices, especially wearables, can measure temperature, pulse, oxygen saturation, blood pressure, etc., among other things.

A further and second aspect of the present invention relates to the use of the method described above for the treatment, prevention and/or aftercare of sleep disorders, exhaustion, stress, pain, degenerative diseases, inflammation, in particular for the prevention and aftercare of the aforementioned health disorders. Furthermore, the method described is used to treat bone fracture healing, wound healing, circulatory and metabolic disorders and/or is used as a supplement to increase performance and improve regeneration in sport, especially competitive sport.

A further and third aspect of the present invention relates to an device for carrying out the method described above. In particular, the device can generate low-frequency current pulses and thus energize an applicator with at least one transmitting coil in order to generate the electromagnetic fields.

In addition, it has proven to be advantageous if the applicator comprises at least one measuring means that can detect the at least one control parameter described above and transmit it to the device or the control parameter input of the device.

Furthermore, according to a further development, the device can be used for the treatment, prevention and/or aftercare of sleep disorders, exhaustion, stress, burn-out, pain, degenerative diseases, inflammation, bone fractures, improvement of wound healing, bone fracture healing, wound healing, circulatory disorders, metabolic disorders, performance enhancement and improvement of regeneration in sports, especially competitive sports.

In the following, with reference to the accompanying drawing, an embodiment of the present invention is described in detail. In the figures:

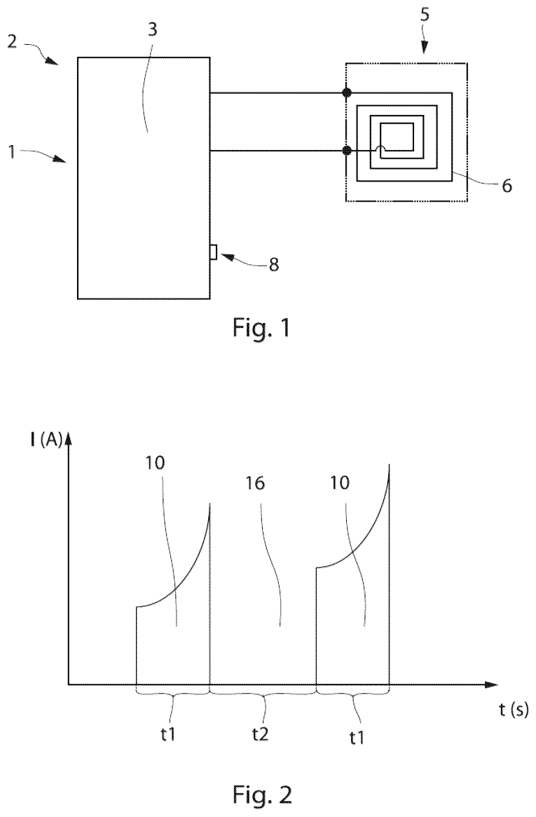

FIG. 1 shows a schematic and exemplary structure of the therapy system with a device for generating low-frequency current pulses and an applicator with a transmitter coil for treating body regions with electromagnetic fields,

FIG. 2 idealized current pulses with a current pulse pause in between,

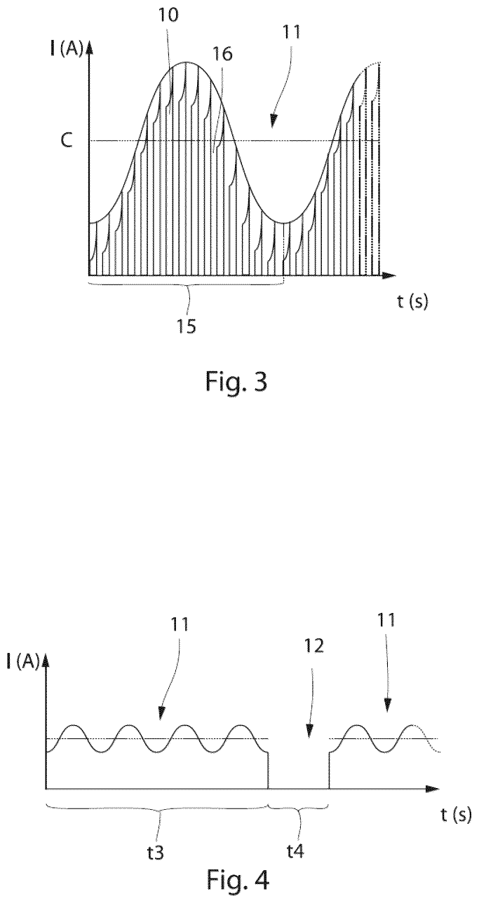

FIG. 3 shows an idealized current pulse sequence with a sinusoidal amplitude modulation of the current pulses, and

FIG. 4 two current pulse sequences, whereby a current pulse sequence pause is provided between the current pulse sequences to regenerate the tissue.

Identical or functionally identical parts or features are identified with the same reference signs in the following detailed description of the figures. Furthermore, not all identical or functionally identical parts or features are given a reference number in the figures.

FIG. 1 shows a preferred and exemplary embodiment of a therapy system 2. The therapy system 2 comprises a device 1 for generating low-frequency current pulses 10 for an applicator 5 and the applicator 5.

The device 1 for generating low-frequency current pulses 10 comprises a generator (not shown in detail) and can be connected to the applicator 5 via a suitable electrical connection.

When used as intended, the applicator 5 can be positioned on, under, around and/or adjacent to a body region of a living being, in particular a human, whereby when energized by the device 1 with the current pulses 10, the applicator generates electromagnetic fields which can act on the body region.

The generator can also be referred to as a signal generator and can generate a large number of current pulses 10, which are shown as an example in FIG. 2 as an amplitude-time diagram. The amplitude A is plotted as current I on the abscissa and the time t on the ordinate.

The generator may comprise one or more oscillators for generating a carrier frequency and one or more RF preamplifiers and main amplifiers configured to generate the current pulses 10. Typically, the generator comprises an oscillator for generating the carrier frequency and an oscillator for generating the modulation frequency. Furthermore, the generator can comprise a signal generator and/or an amplitude control regulator as well as a high-frequency preamplifier and/or a high-frequency main amplifier.

The respective current pulse 10 has a duration t1 and there is a current pulse pause 16 between two current pulses 10. The current pulse pause 16 has a duration t2. The duration t1 of the current pulses 10 can be longer than the duration t2 of the current pulse pauses 16. Preferably, the ratio between t1 and t2 is approx. 8:1.

The respective current pulse 10 has a carrier frequency between 100 Hz and 100 KHz, alternatively between 50 MHz and 250 MHz, whereby the carrier frequency is preferably approx. 150 MHz. Together with the sinusoidal amplitude modulation of the current pulses 10, a particularly focused field of action and a deep penetration depth with a high degree of energy transmission in the body region can be achieved.

The device 1 generates the current pulses 10 at a low frequency. According to the present invention, “low frequency” is understood to mean a frequency at which the current pulses 10 are generated, which is preferably between 100 Hz and 1000 Hz.

The respective current pulse 10 preferably has a duration of between 0.1 ms and 10 s and has at least a first signal component that is in the form of a rectangular current pulse. Preferably, the respective current pulse 10 can be formed from a superposition of a first signal component and at least one second signal component, with the first signal component being a rectangular current pulse and the second signal component having a current that increases or decreases (not shown) linearly or exponentially with time.

A large number of the current pulses 10 form a signal curve, which is shown, for example, in FIGS. 3 and 4.

FIG. 3 in particular shows that the current pulses 10 are sinusoidally amplitude-modulated, according to which the amplitude A of the current pulses 10 rises and falls over time in a sinusoidal curve.

In FIGS. 2 and 3, the sinusoidal curve results from the imaginary connections between the maximum amplitude A of the respective current pulse 10.

In the embodiment example shown, the first signal component is amplitude-modulated, while the second signal component remains constant.

According to an alternative and not shown embodiment, the first signal component can be kept constant while the second signal component is amplitude-modulated.

According to a further alternative and not shown embodiment, the first signal component and the second signal component may be amplitude-modulated, whereby it is further preferred that both signal components are amplitude-modulated equally.

The amplitudes A in the signal curve S are modulated in such a way that the current pulses do not change polarity. In other words, the amplitude A during a current pulse 10 is ≥0 at all times. In particular, it is preferable if the amplitude A is >0 at all times. In this case, the first signal component is always >0.

The amplitude modulation takes place at a modulation frequency of 0.5 to 120 Hz, whereby the modulation frequency is preferably selectable. For this purpose, as shown in FIG. 1, the device 1 can have at least one control parameter input 8, which in the simplest case can be formed by a switch that allows the modulation frequency to be selected. Such a switch can, for example, be a rotary or slide control that can be set to any value between 0.5 Hz and 120 Hz, preferably continuously.

Between the current pulses 10 in the so-called current pulse pause 16, a base current (not shown) can be output by the generator, whereby the base current is many times smaller than the first signal component. Preferably, the base current is a maximum of 30%, preferably 20%, and even more preferably approx. 10% of the current of the first signal component.

The device outputs a current pulse sequence 11, whereby this is formed from a plurality of current pulses 10 and the current pulse sequence 11 describes at least three complete sinusoidal periods with the same amplitude. Preferably, each complete period comprises at least four current pulses 10.

At this point it is noted that the current pulse sequence 11 has at least three complete periods with the same amplitude, but the number of periods does not have to be an integer. However, it is preferable if the respective period begins with a local minimum of the amplitude A and ends with a local minimum of the amplitude.

As can be seen in particular from FIG. 4, the current pulse sequences 11 are interrupted by a current pulse sequence pause 12, whereby a preferred configuration of the signal curve provides that the current pulse sequence has a length of approx. 0.1 ms to approx. 10 s. A current pulse sequence pause can be provided between two current pulse sequences, whereby the current pulse sequence pause 12 is preferably shorter than the current pulse sequence. Preferably, all current pulse sequences last the same length.

The current pulses 10 are transmitted to the applicator 5 via the electrical connections and the transmitter coil 6 generates electromagnetic fields that can be applied to the body regions of a living being.

As shown in FIG. 1, the transmitting coil 6 can be a flat coil, whereby it is particularly preferred if the transmitting coil 6 is an air-core coil. In particular, it is preferable if the transmitting coil 6 has a particularly low inherent inductance. For example, the transmitting coil 6 can be a copper coil.

Instead of or in addition to the embodiment example described above, the device 1 can have at least one control parameter input 8, through which control parameters such as blood pressure, body temperature, pulse, blood sugar value, etc. can be received. Depending on the measured control parameter, the amplitude A of the current pulses 10, the duration t1, the duration t2, the modulation frequency, the modulation amplitude, a duration t4 of the current pulse sequence pause 12 and/or a duration t3 of the current pulse sequence 11 or the number of periods of a current pulse sequence 11 can be set. For example, the control parameter input 8 can comprise a standardized interface that can be connected to a corresponding at least one measuring device or a smart device, such as a wearable, in particular a smart watch, whereby the values recorded by the measuring device are used as control parameters.

In addition to or as an alternative to the at least one measuring device already mentioned, a measuring device may also be provided in the applicator 5, wherein the measuring device in the applicator may be formed, for example, by a receiving coil which comprises the bioreaction of the body region to be treated.

In the treatment, therapy, prevention and/or aftercare of body regions of living beings, such as humans and/or animals, the generated electrometric field can be used for the treatment of sleep disorders, exhaustion, stress, burn-out, pain, degenerative diseases, inflammation, bone fractures, improvement of wound healing, bone fracture healing, wound healing, circulatory disorders, metabolic disorders, performance enhancement and improvement of regeneration in sports, especially competitive sports. For the sake of completeness, it should be noted that this list is not exhaustive.

### LIST OF REFERENCE SIGNS

1 Device
2 Therapy system
3 Generator
5 Applicator
6 Transmitting coil
8 Control parameter input
10 Current pulse
11 Current pulse sequence
12 Current pulse sequence pause
15 Period
16 Current pulse pause
t1 Duration of 10
t2 Duration of 16
t3 Duration of 11
t4 Duration of 12

1. A method (1) for generating electromagnetic fields by an applicator (5) with low-frequency current pulses (10), wherein a plurality of current pulses (10) are generated which follow a signal curve,
wherein the respective current pulse (10) comprises a first signal component in the form of a rectangular pulse,
wherein a current pulse pause (16) is provided between two current pulses (10), and
wherein an amplitude of the current pulses (10) is modulated with a modulation frequency in a sinusoidal signal curve.

2. The method according to claim 1, characterized in that the signal curve comprises a current pulse sequence (11), and in that the current pulse sequence (11) comprises at least three complete sinusoidal periods of approximately equal amplitude.

3. The method according to claim 1, characterized in that the at least one complete sinusoidal period (15) comprises at least four, eight, or at least twelve or more current pulses (10).

4. The method according to claim 1, characterized in that the current pulses (10) are generated with a carrier frequency of 50 MHz to 250 MHz or 100 Hz to 100 KHz.

5. The method according to any of the preceding claims, claim 1, characterized in that the respective current pulse (10) is formed from a superposition of the first signal component and a second signal component, whereby the second signal component being formed from a rising or falling current.

6. The method according to claim 5, characterized in that the second signal component can correspond to a rising or falling current in the form of a linear, exponential function or a Fibonacci number sequence.

7. The method according to claim 1, characterized in that a current pulse sequence pause (12) is provided between two current pulse sequences (11), and in that the current pulse sequence pause (12) has a pause duration (t4) of 0.1 ms to 10 s.

8. The method according to claim 1, characterized in that all current pulses (10) have an amplitude (A) that is selected so that the current pulses (10) do not change polarity in the signal curve.

9. The method according to claim 1, characterized in that all current pulses (10), preferably over at least one current pulse sequence, have an amplitude A>0.

10. The method according to claim 1, characterized in that all current pulses (10), preferably over at least one current pulse sequence, have an amplitude A<0.

11. The method according to claim 1, characterized in that the modulation frequency is between 0.5 and 120 Hz.

12. The method according to claim 1, characterized in that the respective current pulse (10) has a duration of between 0.1 ms and 10 s, preferably 0.5 ms.

13. The method according to claim 1, characterized in that the respective current pulse pause (16) has a duration (t2) of between 0.1 ms and 10 s, preferably 4.5 ms.

14. The method according to claim 1, characterized in that the duration (t1) of a current pulse (10) together with the duration (t2) of a current pulse pause (16) has a frequency of between 3 Hz and 3 KHz over the duration of at least one current pulse sequence (11).

15. The method according to claim 1, characterized in that a control parameter input is provided, and in that at least one control parameter can be provided via the control parameter input, which control parameter determines the amplitude A of the current pulses (10), the modulation frequency, the modulation amplitude, the pause duration (11), a duration (t4) of the current pulse sequence pause (11) and/or, a duration (t3) of the current pulse sequence (11) or number of periods (15).

16. Use of the method according to claim 1, for the treatment, prevention and/or aftercare of sleep disorders, exhaustion, stress, burn-out, pain, degenerative diseases, inflammation, bone fractures, improvement of wound healing, bone fracture healing, wound healing, circulatory disorders, metabolic disorders, performance enhancement and improvement of regeneration in sports, especially competitive sports.

17. A device (1) for carrying out the method according to claim 1, for generating electromagnetic fields.

18. A device (1) according to claim 17 for the treatment, prevention and/or aftercare of sleep disorders, exhaustion, stress, burn-out, pain, degenerative diseases, inflammations, bone fractures, wound healing disorders, circulatory disorders, metabolic disorders, prevention, aftercare and/or regeneration, in particular in competitive sports.
